(19) United States
(12) Patent Application Publication
(10) Pub. No.: US 2004/0266823 A1
(43) Pub. Date: Dec. 30, 2004

(54) Novel piperidine derivatives as modulators of chemokine receptors

(76) Inventors: John Cumming, Macclesfield, Cheshire (GB); Howard Tucker, Macclesfield, Cheshire (GB)

Correspondence Address: FISH & RICHARDSON P.C., 225 FRANKLIN STREET, BOSTON, MA, 02110, US

(21) Appl. No.: 10/495,410

(22) PCT Filed: Nov. 12, 2002
(86) PCT No.: PCT/SE02/02056

(30) Foreign Application Priority Data
Nov. 16, 2001 (GB) 0127547.8

(51) Int. Cl. A61K 31/445; C07D 211/56; C 07D 4 5/02; C 07D 4 9/02

(52) U.S. Cl. 514/317; 514/326; 546/207; 546/224

(57) ABSTRACT

Compounds of the invention, for example compounds of formula (I): compositions comprising them, processes for preparing them and their use in medical therapy (for example modulating CCR5 receptor activity in a warm blooded animal).

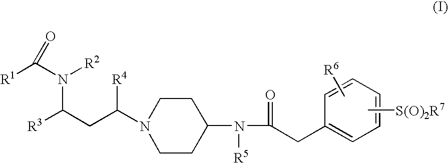

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SE01/01053, EP-A1-1013276, WO00/08013, WO99/38514 and WO99/04794.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1α (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANIES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I):

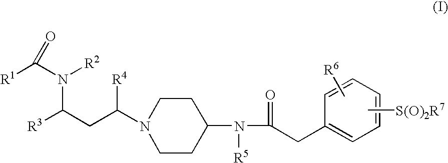

wherein:

R¹ is NHR⁸, C₁₋₆ alkyl {optionally substituted with hydroxy or halo (for example fluoro) or phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, an N-linked 5- or 6-membered non-aromatic heterocyclic ring, or a non-aromatic, 5- or 6-membered mono-heteroatom heterocycic ring, the heteroatom being oxygen or sulphur {optionally substituted by C₁₋₄ alkyl};

R² is hydrogen or C₁₋₆ alkyl;

R³ is phenyl or heteroaryl, either of which is optionally substituted by halo, C₁₋₄ alkyl, C₁₋₄ alkoxy, S(O)ₙ(C₁₋₄ alkyl), nitro, cyano or CF₃; or R³ is C₅₋₇ cycloalkyl;

R⁴ is hydrogen or C₁₋₄ alkyl;

R⁵ is ethyl, allyl or cyclopropyl;

R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;

k, m and n are, independently, 0, 1 or 2;

R⁷ is C₁₋₆ alkyl;

R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₃₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; or a pharmaceutically acceptable salt thereof or a solvate thereof; provided that when R¹ is optionally substituted alkyl, optionally substituted phenyl, optionally substituted heteroaryl [wherein heteroaryl is pyrrolyl, imidazolyl, pyrazolyl, 1,2,3-triazolyl, 1,2,4triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzo[b]furyl, benzo[b]thienyl, phthalazinyl, indanyl, oxadiazolyl or benzthiazolyl] or N-linked pyrrolidinyl, and R² and R⁴ are both hydrogen then R³ is not unsubstituted phenyl; and that when R² is hydrogen, R⁴ is methyl and R³ is unsubstituted phenyl then R¹ is not para-chlorophenyl.

Certain compounds of the present invention can exist in different isomeric forms (such as enantiomers, diastereomers, geometric isomers or tautomers [for example tautomerism between oxo and hydroxy forms, such as on a heteroaryl ring]). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate; or additionally, trifluoroacetate. In one aspect there is provided a compound of the invention which is in the form of a trifluoroacetate or hydrochloide salt.

The compounds of the invention may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl.

Halo includes fluoro, chloro, bromo and iodo; but is preferably fluoro or chloro.

Cycloalkyl is, for example, cyclopropyl, cyclopentyl or cyclohexyl.

N-Linked 5- or 6-membered non-aromatic heterocyclic rings may include a second heteroatom (such as another nitrogen atom or an oxygen or sulphur atom). Examples include piperidinyl, morpholinyl, pyriolidinyl, piperazinyl or thiomorpholinyl. The sulphur of thiomorpholinyl can be oxidised to an S-oxide or S-dioxide.

Non-aromatic, 5- or 6-membered mono-heteroatom heterocyclic ring, the heteroatom being oxygen or sulphur is, for example, tetrahydropyran or tetrahydrothiopyran. Such a group is optionally substituted by, for example, one or two C₁₋₄ alkyl groups.

Heteroaryl is, unless specified otherwise, an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, pyridinyl, pyrimidinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example lH-pyrazolo[3,4b]pyridinyl), quinolinyl, isoquinolinyl, a naphthyridinyl (for example [1,6]naphthyridinyl or [1,8]naphthyridinyl), a benzothiazinyl or dibenzothiophenyl (also known as dibenzothienyl); or an N-oxide thereof, or an S-oxide or S-dioxide thereof. Additionally heteroaryl is [1,2,3]-triazolyl, imidazo[2,1-b]thiazolyl, imidazo[1,2]pyrimidinyl or pyrrolo[2,3-c]pyridinyl. Heteroaryl is, for example, pyridinyl, pyrimidinyl or benzimidazolyl.

In one aspect the present invention provides a compound of formula (I) wherein: R¹ is NHR⁸, C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, or an N-linked 5- or 6-membered non-aromatic heterocyclic ring; R² is hydrogen or C₁₋₆ alkyl; R³ is phenyl or heteroaryl, either of which is optionally substituted in the ortho or meta position by halo, C₁₋₄ alkyl, C₁₋₄ alkoxy, S(O)ₙ(C₁₋₄ alkyl), nitro, cyano or CF₃; or R³ is C₅₋₇ cycloalkyl; R⁴ is hydrogen or C₁₋₄ alkyl; R⁵ is ethyl, allyl or cyclopropyl; R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; k, m and n are, independently, 0, 1 or 2; R⁷ is C₁₋₆ alkyl; R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₅₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; or a pharmaceutically acceptable salt thereof or a solvate thereof; provided that when R¹ is optionally substituted alkyl, optionally substituted phenyl, optionally substituted heteroaryl [wherein heteroaryl is pyrrolyl, imidazolyl, pyrazolyl, 1,2,3-triazolyl, 1,2,4-triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzofuryl, benzo[b]thienyl, phthalazinyl, indanyl, oxadiazolyl or benzthiazolyl] or N-linked pyrrolidinyl, and R² and R⁴ are both hydrogen then R³ is not unsubstituted phenyl; and that when R² is hydrogen, R⁴ is methyl and R³ is unsubstituted phenyl then R¹ is not para-chlorophenyl.

A compound of the invention wherein R¹ is NHR⁸, C₁₋₆ alkyl {optionally substituted with phenyl which is itself optionally substituted by halo}, phenyl {optionally substituted by halo}, heteroaryl {optionally substituted by halo}, or an N-linked 5- or 6-membered non-aromatic heterocyclic ring (such as piperidinyl, pyrrolidinyl or morpholinyl); wherein R⁸ is C₁₋₆ alkyl {optionally substituted with phenyl which is itself optionally substituted by halo} or phenyl {optionally substituted by halo}. Heteroaryl is, for example, pyridinyl or benzimidazolyl.

In a further aspect R¹ is NHR⁸, wherein R⁸ is as defined above (for example R⁸ is C₃₋₇ cycloalkyl, such as cyclopentyl), or R¹ is N-linked piperidinyl, N-linked morpholinyl, tetrahydropyran, tetrahydrothiopyran or C₁₋₄ fluoroalkyl having one to six fluorine atoms.

In another aspect the invention provides a compound of the invention wherein R¹ is NHR⁸, wherein R⁵ is as defined above. R⁸ is, for example, C₃₋₇ cycloalkyl such as cyclopentyl.

In yet another aspect R¹ is phenyl mono-substituted by fluoro, CF₃, S(O)₂CH₃ or NHS(O)₂CH₃; and R³ is mono-fluoro phenyl.

In a further aspect the invention provides a compound of the invention wherein R¹ is N-linked piperidinyl, N-linked morpholinyl, tetrahydropyran, tetrahydrothiopyran or C₁₋₄ fluoroalkyl having one to six fluorine atoms. In a still further aspect R¹ is N-linked piperidinyl or N-linked morpholinyl. In another aspect the invention provides a compound wherein R¹ is tetrahydropyran or tetrahydrothiopyran. In a still further aspect the invention provides a compound wherein R¹ is C₁₋₄ fluoroalkyl having one to six, such as two to three, fluorine atoms. In another aspect R¹ is C₂₋₄ trifluoroalkyl comprising a CF₃ group. Fluoroalkyl is, for example, CF₃CH₂ or CF₃CH₂CH₂.

In a further aspect the invention provides a compound wherein R² is hydrogen or C₁₋₄ alkyl (such as methyl). R² is, for example, hydrogen.

In one aspect the phenyl or heteroaryl ring of R³ is optionally substituted in the ortho or meta position relative to the position of attachment of that ring to the structure of formula (I). In another aspect the invention provides a compound of the invention wherein R³ is phenyl {substituted in the ortho or meta position by halo, C₁₋₄ alkyl, C₁₋₄ alkoxy, S(O)ₙ(C₁₋₄ alkyl), nitro, cyano or CF₃}, heteroaryl {optionally substituted in the ortho or meta position by halo, C₁₋₄ alkyl, C₁₋₄ alkoxy, S(O)ₙ(C₁₋₄ alkyl), nitro, cyano or CF₃} or C₅₋₇ cycloalkyl; n is 0, 1 or 2.

A compound of the invention wherein R³ is phenyl {optionally substituted in the ortho or meta position (for example in the meta position) by halo (for example chloro or fluoro)}, thienyl or cyclohexyl.

In a still further aspect R³ is phenyl optionally substituted (such as un-substituted or mono-substituted) by halo (such as chloro or fluoro; for example fluoro).

In another aspect of the invention R³ is phenyl or 3-fluorophenyl.

In a further aspect the carbon to which R³ is attached has the S absolute configuration. In a still further aspect the carbon to which R³ is attached has the R absolute configuration.

A compound of the invention wherein R⁴ is hydrogen or methyl. In a further aspect of the invention R⁴ is methyl. In a still further aspect of the invention R⁴ is hydrogen. In another aspect when R⁴ is C₁₋₄ alkyl (such as methyl) the carbon to which R⁴ is attached has the R absolute configuration.

A compound of the invention wherein R⁵ is ethyl.

In a still further aspect of the invention R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; and m is as defined above.

A compound of the invention wherein R⁶ is halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; and m is as defined above.

A compound of the invention wherein R⁶ is hydrogen.

A compound of the invention wherein R⁶ is hydrogen, halo, hydroxy, nitro, cyano, C₁₋₄ alkyl, C₁₋₄ alkoxy, CF₃ or OCF₃.

A compound of the invention wherein R⁶ is halo, hydroxy, nitro, cyano, C₁₋₄ alkyl; C₁₋₄ alkoxy, CF₃ or OCF₃.

A compound of the invention wherein R⁷ is C₁₋₄ alkyl. For example R⁷ is methyl. A compound of the invention wherein the S(O)₂R⁷ group of formula (I) is para disposed to the remainder of the structure of formula (I), that is, it is as shown here:

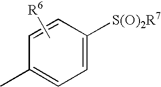

The variables k, m and n are, for example, 0 or 2, for example they are all 2.

In another aspect the present invention provides a compound of formula (I):

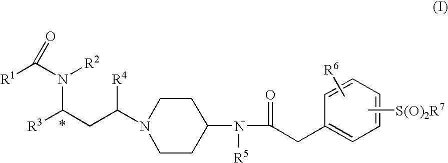

wherein the compound has the S absolute configuration at chiral centre marked with an asterisk ‘*’; and

R¹ is NHR⁸, C₁₋₆ alkyl {optionally substituted with hydroxy or halo (for example fluoro) or phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, an N-linked 5- or 6-membered non-aromatic heterocyclic ring, or a non-aromatic, 5- or 6-membered mono-heteroatom heterocyclic ring, the heteroatom being oxygen or sulphur {optionally substituted by C₁₋₄ alkyl};

R² is hydrogen or C₁₋₆ alkyl;

R³ is phenyl or heteroaryl, either of which is optionally substituted by halo, C₁₋₄ alkyl, C₁₋₄

alkoxy, S(O)ₙ(C₁₋₄ alkyl), nitro, cyano or CF₃; or R³ is C₅₋₇ cycloalkyl;

R⁴ is hydrogen or C₁₋₄ alkyl;

R⁵ is ethyl, allyl or cyclopropyl;

R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;

k, m and n are, independently, 0, 1 or 2;

R⁷ is C₁₋₆ alkyl;

R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₃₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};

or a pharmaceutically acceptable salt thereof or a solvate thereof.

In a still further aspect the present invention provides a compound of formula (I) wherein the compound has S absolute configuration at chiral centre marked with an asterisk ‘*’; and

R¹ is NHR⁸, C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, or an N-linked 5- or 6-membered non-aromatic heterocyclic ring;

R² is hydrogen or C₁₋₆ alkyl;

R³ is phenyl or heteroaryl, either of which is optionally substituted in the ortho or meta position by halo, C₁₋₄ alkyl, C₁₋₄ alkoxy, S(O)ₙ(C₁₋₄ alkyl), nitro, cyano or CF₃; or R³ is C₅₋₇ cycloalkyl;

R⁴ is hydrogen or C₁₋₄ alkyl;

R⁵ is ethyl, allyl or cyclopropyl;

R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;

k, m and n are, independently, 0, 1 or 2;

R⁷ is C₁₋₆ alkyl;

R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₃₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF_{3};}

or a pharmaceutically acceptable salt thereof or a solvate thereof.

In a further aspect the present invention provides a compound of formula (Ia):

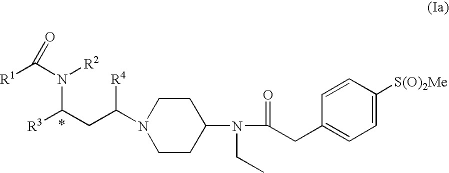

wherein R¹, R², R³ and R⁴ are as defined above; provided that when R¹ is optionally substituted alkyl, optionally substituted phenyl, optionally substituted heteroaryl [wherein heteroaryl is pyrrolyl, imidazolyl, pyrazolyl, 1,2,3-triazolyl, 1,2,4-triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzo[b]furyl, benzo[b]thienyl, phthalazinyl, indanyl, oxadiazolyl or benzthiazolyl] or N-linked pyrrolidinyl, and R² and R⁴ are both hydrogen then R³ is not unsubstituted phenyl; and that when R² is hydrogen, R⁴ is methyl and R³ is unsubstituted phenyl then R¹ is not para-chlorophenyl.

In a still further aspect the present invention provides a compound of formula (Ia) wherein the compounds of formula (Ia) have the S absolute configuration at chiral centre marked with an asterisk ‘*’; and wherein R¹, R², R³ and R⁴ are as defined above.

The following compounds illustrate the invention.

TABLE I
Table I comprises compounds of formula (Ia).

| Compound | | | | | | | LCMS |
|---|---|---|---|---|---|---|---|
| No. | R¹ | R² | R³ | R⁴ | * Chirality | Adduct | (MH+) |
| 1 | 6-Chloro-3-pyridinyl | H | Phenyl | H | S isomer | | 597 |
| 2 | 2-Chloro-4-pyridinyl | H | Phenyl | H | S isomer | | 597 |
| 3 | Benzimidazol-5-yl | H | Phenyl | H | S isomer | | 602 |
| 4 | 1-Phenyl-1-methylethyl | H | Phenyl | H | S isomer | | 604 |
| 5 | 1-(4-Chlorophenyl)-1-methylethyl | H | Phenyl | H | S isomer | | 638 |
| 6 | (1S)-1-Phenyl-2-methylprop-1-yl | H | Phenyl | H | S isomer | | 618 |
| 7 | (1S)-1-Phenyleth-1-yl | H | Phenyl | H | S isomer | | 590 |
| 8 | 1-(4-Chlorophenyl)ethyl | H | Phenyl | H | S isomer | | 624 |
| 9 | 1-(4-Fluorophenyl)ethyl | H | Phenyl | H | S isomer | | 608 |
| 10 | 2,2-Dimethylpropyl | H | Phenyl | H | S isomer | | 556 |
| 11 | Phenyl | H | Phenyl | H | S isomer | | 562 |
| 12 | 2-Fluorophenyl | H | Phenyl | H | S isomer | | 580 |
| 13 | 3,4-Difluorophenyl | H | Phenyl | H | S isomer | | 598 |
| 14 | 3-Fluorophenyl | H | Phenyl | H | S isomer | | 580 |
| 15 | 4-Chlorophenyl | H | Phenyl | H | R isomer | | 596 |
| 16 | 4-Chlorophenyl | H | Phenyl | H | S isomer | | 596 |
| 17 | 4-Fluorophenyl | H | Phenyl | H | S isomer | | 580 |
| 18 | Piperidin-1-yl | H | Phenyl | H | | | 569 |
| 19 | Piperidin-1-yl | H | Phenyl | H | S isomer | | 569 |
| 20 | Pyrrolidin-1-yl | H | Phenyl | H | | | 555 |
| 21 | Morpholin-4-yl | H | Phenyl | H | | | 571 |
| 22 | Morpholin-4-yl | H | Phenyl | H | S isomer | | 571 |
| 23 | 2-Phenylethylamino | H | Phenyl | H | | | 605 |
| 24 | 1-Phenylethylamino | H | Phenyl | H | | | 605 |
| 25 | Phenylmethylamino | H | Phenyl | H | | | 591 |
| 26 | Ethylamino | H | Phenyl | H | | | 529 |
| 27 | iso-propylamino | H | Phenyl | H | | | 543 |
| 28 | Phenylamino | H | Phenyl | H | | | 577 |
| 29 | 3-Chlorophenylamino | H | Phenyl | H | | | 611 |
| 30 | 4-Chlorophenylamino | H | Phenyl | H | | | 611 |
| 31 | Propylamino | H | Phenyl | H | | | 543 |
| 32 | tert-butylamino | H | Phenyl | H | | | 557 |
| 33 | 4-Chlorophenyl | H | Phenyl | Methyl | S isomer | | 610 |
| 34 | Piperidin-1-yl | H | Thien-2-yl | H | | | 575 |
| 35 | Pyrrolidin-1-yl | H | Thien-2-yl | H | | | 561 |
| 36 | Morpholin-4-yl | H | Thien-2-yl | H | | | 577 |
| 37 | 4-Chlorophenyl | H | Cyclohexyl | H | | | 602 |
| 38 | Phenyl | Methyl | Phenyl | H | | | 576 |
| 39 | 4-Chlorophenyl | Methyl | Phenyl | H | | | 610 |
| 40 | Morpholin-4-yl | Methyl | Phenyl | H | | | 585 |
| 41 | Cyclohexylamino | H | Phenyl | H | S isomer | | 583 |
| 42 | Cyclohexylamino | H | Thien-2-yl | H | | | 589 |
| 43 | Cyclohexylamino | H | Cyclohexyl | H | | | 589 |
| 44 | 5-Methylisoxazol-4-yl | H | Phenyl | H | S isomer | | 567 |
| 45 | Pyrazol-3-yl | H | Phenyl | H | S isomer | | 552 |
| 46 | Thiazol-4-yl | H | Phenyl | H | S isomer | | 569 |
| 47 | 2-Methylimidazol-5-yl | H | Phenyl | H | S isomer | | 566 |
| 48 | 2-Methyloxazol-4-yl | H | Phenyl | H | S isomer | | 567 |
| 49 | Isothiazol-5-yl | H | Phenyl | H | S isomer | | 569 |
| 50 | [1,2,4]-Triazol-5-yl | H | Phenyl | H | S isomer | | 553 |
| 51 | Thiazol-5-yl | H | Phenyl | H | S isomer | | 569 |
| 52 | Furan-3-yl | H | Phenyl | H | S isomer | | 552 |
| 53 | Pyrrol-2-yl | H | Phenyl | H | S isomer | | 551 |
| 54 | Phenyl | H | Phenyl | Methyl | S isomer | | |
| 55 | 2-Fluorophenyl | H | Phenyl | Methyl | S isomer | | |
| 56 | 3,4-Difluorophenyl | H | Phenyl | Methyl | S isomer | | |
| 57 | 4-Fluorophenyl | H | Phenyl | Methyl | S isomer | | |
| 58 | Piperidin-1-yl | H | Phenyl | Methyl | S isomer | | |
| 59 | Pyrrolidin-1-yl | H | Phenyl | Methyl | S isomer | | |
| 60 | Morpholin-4-yl | H | Phenyl | Methyl | S isomer | | |
| 61 | Cyclopentylamino | H | Phenyl | H | S isomer | | 568 |
| 62 | Isothiadiazol-3-yl | H | Phenyl | H | S isomer | hydrochloride | 570 |
| 63 | [1,2,3]-Thiadiazol-4-yl | H | Phenyl | H | S isomer | trifluoroacetate | 570 |
| 64 | Isoxazol-5-yl | H | Phenyl | H | S isomer | trifluoroacetate | 553 |
| 65 | 4-Methyl-5-acetyl-pyrazol-3-yl | H | Phenyl | H | S isomer | | 608 |
| 66 | 3-Carbomethoxypyrazin-2-yl | H | Phenyl | H | S isomer | | 622 |
| 67 | 5-Methylfuran-2-yl | H | Phenyl | H | S isomer | | 566 |
| 68 | 6-Acetylaminopyridin-3-yl | H | Phenyl | H | S isomer | hydrochloride | 620 |
| 69 | 5-Acetylthien-2-yl | H | Phenyl | H | S isomer | | 610 |
| 70 | 1-Methyl-4-chloropyrazol-5-yl | H | Phenyl | H | S isomer | | 601 |
| 71 | 4-Methylpyridin-2-yl | H | Phenyl | H | S isomer | | 577 |
| 72 | 5-Oxo-5,6-dihydroimidazo[1,2- | H | Phenyl | H | S isomer | | 619 |
| | c]pyrimidin-2-yl | | | | | | |
| 73 | 1H-Pyrrolo[2,3-c]pyridin-2-yl | H | Phenyl | H | S isomer | | 602 |
| 74 | 1,5-Dimethylpyrazol-3-yl | H | Phenyl | H | S isomer | trifluoroacetate | 580 |
| 75 | 4,6-Dimethoxypyrimidin-5-yl | H | Phenyl | H | S isomer | trifluoroacetate | 624 |
| 76 | Imidazo[2,1-b]thiazol-6-yl | H | Phenyl | H | S isomer | trifluoroacetate | 608 |
| 77 | 2-Methanesulfanylpyrimidin-4-yl | H | Phenyl | H | S isomer | trifluoroacetate | 610 |
| 78 | 1,3-Dimethylpyrazol-5-yl | H | Phenyl | H | S isomer | trifluoroacetate | 580 |
| 79 | 5-Methanesulfonylthien-2-yl | H | Phenyl | H | S isomer | | 646 |
| 80 | 3-Cyano-4-acetyl-5-methylpyrrol-2-yl | H | Phenyl | H | S isomer | trifluoroacetate | 632 |
| 81 | 2,2-Dimethyltetrahydropyran-4-yl | H | Phenyl | H | S isomer | | 598 |
| 82 | 3,5-Difluorophenyl | H | Phenyl | H | S isomer | hydrochloride | 598 |
| 83 | Thiomorpholin-4-yl | H | Phenyl | H | S isomer | | 587 |
| 84 | Difluoromethyl | H | Phenyl | H | S isomer | hydrochloride | 536 |
| 85 | 4-Trifluoromethylpyridin-3-yl | H | Phenyl | H | S isomer | hydrochloride | 631 |
| 86 | 2,2,2-Trifluoroethyl | H | Phenyl | H | S isomer | hydrochloride | 568 |
| 87 | 4,4,4-Trifluorobut-2-yl | H | Phenyl | H | S isomer | hydrochloride | 596 |
| 88 | 5-Trifluoromethylfuran-2-yl | H | Phenyl | H | S isomer | hydrochloride | 620 |
| 89 | 6-Oxo-1,6-dihydropyridin-3-yl | H | Phenyl | H | S isomer | | 579 |
| 90 | Imidazol-5-yl | H | Phenyl | H | S isomer | | 552 |
| 91 | 1,1-Dioxothiomorpholin-4-yl | H | Phenyl | H | S isomer | | 619 |
| 92 | 4-Isopropyl-[1,2,3]Triazol-5-yl | H | Phenyl | H | S isomer | hydrochloride | 595 |
| 93 | 3-Cyanophenyl | H | Phenyl | H | S isomer | hydrochloride | 587 |
| 94 | 4-Cyanophenyl | H | Phenyl | H | S isomer | hydrochloride | 587 |
| 95 | Tetrahydropyran-4-yl | H | Phenyl | Methyl | S isomer | | 584 |
| 96 | 3,3,3-Trifluoropropyl | H | Phenyl | H | S isomer | hydrochloride | 582 |
| 97 | 4-Acetylaminophenyl | H | Phenyl | H | S isomer | | 619 |
| 98 | 3-Methanesulfonylphenyl | H | Phenyl | H | S isomer | | 640 |
| 99 | 4-Methanesulfonylphenyl | H | Phenyl | H | S isomer | | 640 |
| 100 | 4-Methylaminosulfonylphenyl | H | Phenyl | H | S isomer | | 655 |
| 101 | 4-Methanesulfonylaminophenyl | H | Phenyl | H | S isomer | | 655 |
| 102 | 4-Trifluoromethylphenyl | H | Phenyl | H | S isomer | | 630 |
| 103 | Phenyl | H | 3-Fluorophenyl | H | S isomer | | 580 |
| 104 | 3-Fluorophenyl | H | 3-Fluorophenyl | H | S isomer | | 598 |
| 105 | Tetrahydropyran-4-yl | H | 3-Fluorophenyl | H | S isomer | | 588 |
| 106 | 2,2-Dimethylpropyl | H | 3-Fluorophenyl | H | S isomer | | 574 |
| 107 | Piperidin-1-yl | H | 3-Fluorophenyl | H | S isomer | | 587 |
| 108 | 2,2,2-Trifluoroethyl | H | 3-Fluorophenyl | H | S isomer | | 586 |
| 109 | Tetrahydrothiopyran-4-yl | H | Phenyl | H | S isomer | | 586 |
| 110 | 2-Methyl-2-hydroxypropyl | H | Phenyl | H | S isomer | hydrochloride | 558 |
| 111 | 2,2,2-Trifluoroethyl | H | Phenyl | Methyl | S isomer | | |
| 112 | 3,3,3-Trifluoropropyl | H | Phenyl | Methyl | S isomer | | |
| 113 | Isothiadiazol-3-yl | H | Phenyl | H | S isomer | | |
| 114 | [1,2,3]-Thiadiazol-4-yl | H | Phenyl | H | S isomer | | |
| 115 | Isoxazol-5-yl | H | Phenyl | H | S isomer | | |
| 116 | 6-Acetylaminopyridin-3-yl | H | Phenyl | H | S isomer | | |
| 117 | 1,5-Dimethylpyrazol-3-yl | H | Phenyl | H | S isomer | | |
| 118 | 4,6-Dimethoxypyrimidin-5-yl | H | Phenyl | H | S isomer | | |
| 119 | Imidazo[2,1-b]thiazol-6-yl | H | Phenyl | H | S isomer | | |
| 120 | 2-Methanesulfanylpyrimidin-4-yl | H | Phenyl | H | S isomer | | |
| 121 | 1,3-Dimethylpyrazol-5-yl | H | Phenyl | H | S isomer | | |
| 122 | 3-Cyano-4-acetyl-5-methylpyrrol-2-yl | H | Phenyl | H | S isomer | | |
| 123 | 3,5-Difluorophenyl | H | Phenyl | H | S isomer | | |
| 124 | Difluoromethyl | H | Phenyl | H | S isomer | | |
| 125 | 4-Trifluoromethylpyridin-3-yl | H | Phenyl | H | S isomer | | |
| 126 | 2,2,2-Trifluoroethyl | H | Phenyl | H | S isomer | | |
| 127 | 4,4,4-Trifluorobut-2-yl | H | Phenyl | H | S isomer | | |
| 128 | 5-Trifluoromethylfuran-2-yl | H | Phenyl | H | S isomer | | |
| 129 | 4-Isopropyl-[1,2,3]Triazol-5-yl | H | Phenyl | H | S isomer | | |
| 130 | 3-Cyanophenyl | H | Phenyl | H | S isomer | | |
| 131 | 4-Cyanophenyl | H | Phenyl | H | S isomer | | |
| 132 | 3,3,3-Trifluoropropyl | H | Phenyl | H | S isomer | | |
| 133 | 2-Methyl-2-hydroxypropyl | H | Phenyl | H | S isomer | | |

In another aspect the present invention provides each individual compound of Table I. In a further aspect the invention provides Compound No. 1, 2, 3, 4, 5, 6, 7, 8, 9, 10, 11, 12, 13, 14, 15, 16, 17, 33, 44, 45, 46, 47, 48, 49, 50, 51, 52 or 53 of Table I, or a pharmaceutically acceptable salt thereof or a solvate thereof. In a still further aspect the invention provides Compound No. 54 to 133 of Table I, or a pharmaceutically acceptable salt thereof or a solvate thereof.

The compounds of the invention can be prepared as shown in the processes on pages marked Schemes 1 to 3 below. (In Schemes 1 to 3 Ac is acetyl; Boc is tert-butoxycarbonyl; Ph is phenyl; and, TFA is trifluoroacetic acid. Suitable coupling agents include PyBrOP (bromo-tris-pyrrolidino-phosphonium hexafluorophosphate) and HATU.)

A compound of the invention can be prepared by coupling a compound of formula (II):

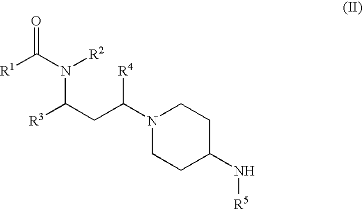

wherein R¹, R², R³, R⁴ and R⁵ are as defined above, with a compound of formula (III):

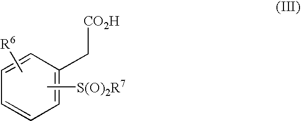

wherein R⁶ and R⁷ are as defined above, in the presence of a suitable coupling agent (for example PyBrOP or HATU) in the presence of a suitable base (such as a tertiary amine, for example diisopropylethylamine) in a suitable solvent (for example N-methylpyrrolidinone or a chlorinated solvent, such as dichloromethane) at room temperature (for example 10-30° C.).

Alternatively, a compound of the invention can be prepared by reacting a compound of formula (IV):

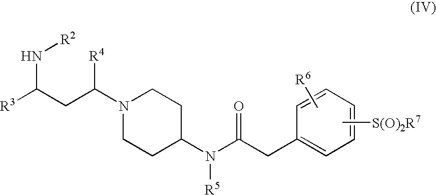

wherein R², R³, R⁴ R⁵, R⁶ and R⁷ are as defined above, with:

a) an acid of formula R¹CO₂H in the presence of a suitable coupling agent (for example PyBrOP or HATU) in the presence of a suitable base (such as a tertiary amine, for example diisopropylethylamine) in a suitable solvent (for example N-methylpyrrolidinone or a chlorinated solvent, such as dichloromethane) at room temperature (for example 10-30° C.);

b) an acid chloride of formula R¹C(O)Cl in the presence of a suitable base (such as a tertiary amine, for example triethylamine or diisopropylethylamine) in a suitable solvent (for example a chlorinated solvent, such as dichloromethane) at room temperature (for example 10-30° C.);

c) an isocyanate of formula R¹NCO in the presence of a suitable base (such as a tertiary amine, for example triethylamine or diisopropylethylamine) in a suitable solvent (for example an ester such as ethyl acetate) at room temperature (for example 10-30° C.); or,

d) a carbamoyl chloride in the presence of a suitable base (such as a tertiary amine, for example triethylamine or dusopropylethylamine);

wherein R¹ is as defined above.

The starting materials for all the processes and Schemes are either commercially available or can be prepared by literature methods, adapting literature methods or by following or adapting Methods herein described.

In a further aspect the invention provides processes for preparing the compounds of the invention. Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HW)) into target calls and, therefore, are of value in the prevention of infection by viruses (such as HIV), the treatment of infection by viruses (such as H) and the prevention and/or treatment of acquired immune deficiency syndrome (AIDS).

According to a further feature of the invention there is provided a compound of the invention, or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to, said animal an effective amount of a compound of the present invention, or a pharmaceutically acceptable salt thereof or a solvate thereof.

The present invention also provides the use of a compound of the invention, or a pharmaceutically acceptable salt thereof or a solvate thereof, as a medicament, especially a medicament for the treatment of transplant rejection, respiratory disease, psoriasis or arthritis (especially rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the invention, or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention also provides a compound of the invention, or a pharmaceutically acceptable salt thereof or a solvate thereof, for use as a medicament, especially a medicament for the treatment of rheumatoid arthritis.

In another aspect the present invention provides the use of a compound of the invention, or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention further provides the use of a compound of the invention, or a pharmaceutically acceptable salt thereof, in the manufacture of a medicament for use in the treatment of:

(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;

(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;

(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;

(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);

(5) (Allograft rejection) acute and chronic following, for example, transplantation of kidney, heart, liver, lung, bone marrow, skin or cornea; or chronic graft versus host disease;

and/or

(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle;

in a warm blooded animal, such as man.

The present invention further provides a method of treating a cheinokihe mediated disease state (especially a CCR5 mediated disease state) in a warm blooded animal, such as man, which comprises administering to a mammal in need of such treatment an effective amount of a compound of the invention, or a pharmaceutically acceptable salt thereof or solvate thereof.

In order to use a compound of the invention, or a pharmaceutically acceptable salt thereof or solvate thereof, for the therape utic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the invention, or a pharmaceutically acceptable salt thereof or a solvate thereof (active ingredient), and a pharmaceutically acceptable adjuvant, diluent or carrier. In a finther aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of the invention, or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glanorgan, UK Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent: gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

DMSO dimethyl sulphoxide;

DMF N-dimethylformamide;

DCM dichloromethane;

THF tetrahdydrofuran;

DIPEA N,N-diisopropylethylamine;

NMP N-methylpyrrolidinone;

HATU O-(7-Azabenzotriazol-1-yl)-N,N′,N′-tetramethyluronium hexafluorophosphate;

HBTU O-(7-Benzotriazol-1-yl)-N,N,N′,N′-tetramethyluronium hexafluorophosphate;

Boc tert-butoxycarbonyl

MeOH methanol;

EtOH ethanol; and

EtOAc ethyl acetate.

### EXAMPLE 1

This Example illustrates the preparation of (S)-N-[1-(3-phenyl-3-benzoylamino]propyl)-4-piperidinyl]-N-ethylmethanesulfonylphenylacetamide (Compound No. 11 of Table I).

To a mixture of (S)-N-[1-(3-phenyl-3-aminopropyl)-4-piperidinyl]-N-ethyl-4-methanesulfonylphenylacetamide dihydrochloride (Method A; 220 mg, 0.42 mmol) and DIPEA (0.75 mL) in DCM (5 mL) was added benzoic acid (100 mg, 0.82 mmol). To the resulting mixture was added HATU (300 mg). The mixture was left at room temperature for 18 h, washed with 2M aqueous sodium hydroxide and water, then evaporated. Purification was achieved by BondElut chromatography eluting with a solvent mixture of ethyl acetate to 20% methanol in ethyl acetate to give the title compound (164 mg); NMR (d⁶-DMSO at 100° C.): 1.1 (t, 3H), 1.5 (m, 2H), 1.75 (m, 2), 2.0 (m, 4H), 2.35 (t, 2H), 2.9 (m, 2H), 3.13 (s, 3H), 3.25 (q, 2H), 3.82 (s, 2H), 3.85 (m, 1H), 5.15 (m, 1H), 7.2-7.5 (m, 10H), 7.85 (m, 4H), 8.52 (d, 1H); MS: 562.

The procedure described in Example 1 can be repeated using different carboxylic acids (such as 2-chloroisonicotinic acid, indole-5-carboxylic acid) in place of benzoic acid or different amines (such as (4′S)-N-[1-(4phenyl-4-aminobut-2-yl)-4-piperidinyl]-N-ethyl-4-methanesulfonylphenylacetamide dihydrochloride (Method D)) in place of (S)-N-[1-(3-phenyl-3-aminopropyl)-4-piperidinyl]-N-ethylmethanesulfonylphenylacetamide dihydrochloride.

### EXAMPLE 2

This Example illustrates the preparation of (S)-N-[1-(3-phenyl-3-[piperidin-1-ylcarboxyamino]propyl)-4-piperidinyl]-N-ethyl-4-methanesulfonylphenylacetamide (Compound No. 19 of Table I).

To a mixture of (S)-N-[1-(3-phenyl-3-aminopropyl)-4-piperdinyl]-N-ethyl-4-methanesulfonylphenylacetamide dihydrochloride (Method A; 200 mg, 0.38 mmol) and triethylamine (0.21 mL) in DCM (10 mL) was added 1-piperidinecarbonyl chloride (47 μL, 0.38 mmol) and the resulting mixture stirred at room temperature for 18 h. The mixture was evaporated and the residue purified by eluting through a 20 g BondElut cartridge giving the title compound (107 mg, 50%); NMR (CDCl₃): 1.2 (t, 1H), 1.25 (m, 3H), 1.4 (t, 1H), 1.6 (m, 7H), 1.8 (m, 3H), 1.9 (m, 5H), 2.3 (m, 1H), 2.6 (m, 1H), 3.0 (s, 3H), 3.4 (m, 6H), 3.8 (m, 2H), 4.9 (m, 1H), 6.3 (m, 1H), 7.25 (m, 5H), 7,45 (d, 2H) 7.9 (d, 2H); MS: 569.

The procedure described in Example 2 can be repeated using different carbamoyl chlorides (such as 4 morpholinecarbonyl chloride and 1-pyrrolidinecarbonyl chloride) in place of 1-piperidinecarbonyl chloride, or different amines (such as N-[1-(3-[2-thienyl]-3-aminopropyl)-4-piperidinyl]-N-ethyl-4-methanesulfonylphenylacetamide (Method G)) in place of (S)-N-[1-(3-phenyl-3-aminopropyl)-4-piperidinyl]-N-ethyl-4-methanesulfonylphenylacetamide dihydrochloride.

### EXAMPLE 3

This Example illustrates the preparation of N-[1-(3-phenyl-3-[3-chlorophenylamino-carboxyamino]propyl)-4-piperidinyl]-N-ethyl-4-methanesulfonylphenylacetamide (Compound No. 29 of Table I).

Step 1: Preparation of N-[1-(3-phenyl-3-Boc-aminopropyl)-4-piperidinyl]-N-ethyl-4-methanesulfonylphenylacetamide

To a solution of 3-phenyl-3-Boc-aminopropionaldehdye (4.6 mg, 18.5 mmol) and N-(4-piperidinyl)-N-ethyl-4-methanesulfonylphenylacetamide (6.0 g, 18.5 mmol) in DCM (100 mL) and methanol (10 mL) was added one drop of acetic acid and the resulting mixture was stirred at room temperature for 1 h. Sodium triacetoxyborohydride (3.9 g, 18.5 mmol) was added and the mixture was stirred at room temperature for 18 h. The reaction mixture was washed with saturated aqueous sodium bicarbonate solution (3×100 mL), dried and evaporated. The residue was purified by silica gel chromatography (eluent: 1:1 ethyl acetate/isohexane then 15% methanol in ethyl acetate giving the sub-titled compound (11 g).

Step 2: Preparation of N-[1-(3-phenyl-3-aminopropyl)-4-piperidinyl]-N-ethyl-4-methanesulfonylphenylacetamide

N-[1-(3-phenyl-3-Boc-aminopropyl)-4-piperidinyl]-N-ethyl-4-methanesulfonylphenylacetamide (11 g) was dissolved in trifluoroacetic acid (50 mL) and the resulting mixture was stirred at room temperature for 2 h. The mixture was evaporated and the residue treated with saturated aqueous sodium bicarbonate solution (150 mL). The resulting mixture was extracted with diethyl ether (2×30 mL). The aqueousphase was evaporated and the residue suspended in methanol (75 mL). The resulting mixture was filtered and the residue washed with methanol. The combined washings and filtrate were evaporated and the residue azeotroped with toluene to give the sub-titled compound (8.4 g).

Step 3: Preparation of Title Compound

To a solution of N-[1-(3-phenyl-3-aminopropyl)-4-piperidinyl]-N-ethyl-4-methanesulfonylphenylacetamide (230 mg, 0.50 mmol) and triethylamine (0.50 mmol) in ethyl acetate (5 mL) was added 3-chlorophenyl isocyanate (77 mg, 0.50 mmol) and the resulting mixture stirred at room temperature for 72 h. The mixture was eluted through a silica gel column with ethyl acetate followed by 5% methanol in ethyl acetate to give the title compound (140 mg, 46%); MS: 611.

The procedure described in Example 3 can be repeated using different isocyanates (such as phenyl isocyanate, ethyl isocyanate and 2-phenylethyl isocyanate) in place of 3-chlorophenyl isocyanate.

### EXAMPLE 4

This Example illustrates the preparation N-[1-(3-cyclohexyl-3-[4-chlorobenzoylamino]propyl)-4-piperidinyl]-N-ethyl-4-methanesulfonylphenylacetamide (Compound No.37 of Table I).

To a solution of N-[1-(3-cyclohexyl-3-aminopropyl)-4-piperidinyl]-N-ethyl-4-methanesulfonylphenylacetamide (Method K, 250 mg, 5.4 mmol) and triethylamine (5.4 mmol) in DCM (10 mL) was added 4-chlorobenzoyl chloride (5.4 mmol) and the resulting mixture was stirred at room temperature for 2 h. Polymer supported isocyanate (200 mg) and trisamine resin (200 mg) were added and the mixture left standing at room temperature for 18 h. The mixture was filtered, washed with saturated aqueous sodium hydrogen carbonate solution (2×20 mL), dried and eluted through a 10 g SCX cartridge with DCM then 10% methanol in DCM then methanol and finally 0.5M ammonia in methanol yielding the title compound as solid (169 mg) after trituration with diethyl ether; NMR: 0.8-2 (m, 25H), 2.2 (m, 2H), 2.85 (m, 2H), 3.25 (s, 3H), 3.8 (m, 3H), 7.25 (m, 4H), 7.8 (m, 4H), 8.05 (d, 1H); MS: 602.

The procedure described in Example 4 can be repeated using different acid chlorides such as benzoyl chloride) in place of 4-chlorobenzoyl chloride or different amines (such as N-[1-(3-phenyl-3-methylaminopropyl)-4-piperidinyl]-N-ethyl-4-methanesulfonylphenylacetamide (Method P)) in place of N-[1-(3-cyclohexyl-3-aminopropyl)-4-piperidinyl]-N-ethyl-4-methanesulfonylphenylacetamide.

### EXAMPLE 5

This Example illustrates the preparation of (S)-N-{1-[3-(3,3,3-trifluoropropionylamino)-3-(3-fluorophenyl)propyl]piperidinyl}-N-ethyl-2-(4-methanesulfonyl-phenyl)acetamide (Compound No. 108 of Table I).

To a stirred solution of 3,3,3-trifluoropropionic acid (32 mg, 0.24 mmol) in DCM (1 mL) was added 1-chloro-N,N,2-trimethyl-1-propenylamine (0.037 mL, 0.23 mmol) and the resulting mixture was stirred at room temperature for 1 h. To this mixture was added a solution of (S)-N-{1-[3-amino-3-(3-fluorophenyl)propyl]piperidinfyl}-N-ethyl-2-(4-methanesulfonyl-phenyl)acetamide (Method R, 100 mg, 0.21 mmol) in DCM (1 mL) and triethylamine (0.1 mL, 0.65 mmol) and the resulting mixture was stirred at room temperature for 18 h. The mixture was diluted with DCM, washed with water and saturated aqueous sodium bicarbonate solution. The organic phase was dried and evaporated and the residue was purified by eluting through a BondElut cartridge (gradient elution DCM to 5% methanol in DCM) giving the title compound as a solid (52 mg); NMR: 1.05 and 1.08 (t, 3H), 1.45 and 1.50 (m, 2H), 1.70 (m, 2H), 1.80 (m, 2H), 1.95 (m, 2H), 2.25 (t, 2H), 2.88 (m, 2H), 3.20 (s, 3H), 3.25 and 3.30 (q, 2H), 3.30 (s, 2H), 3.67 and 4.10 (m, 1H), 3.82 and 3.89 (s, 2H), 4.89 (m, 1H), 7.10 (m, 3H), 7.42 (m, 1H), 7.50 (d, 2H), 7.85 (d, 2H), 8.70 (dd, 1H); MS: 586 (MH+).

The procedure described in Example 5 can be repeated using different carboxylic acids (such as benzoic acid, 3-fluorobenzoic acid, tetrahydropyran-4-carboxylic acid, or 3,3-dimethylbutyric acid) in place of 3,3,3-trifluoropropionic acid.

### EXAMPLE 6

This Example illustrates the preparation of (S)-N-[1-(3-phenyl-3-[1,1-dioxothiomorpholin-4-ylcarboxyamino]propyl)-4-piperidinyl]-N-ethyl-4-methanesulfonylphenylacetamide (Compound No. 91 of Table I).

To a solution of (S)-N-[1-(3-phenyl-3-[thiomorpholin-4-ylcarboxyamino]propyl)-4-piperidinyl]-N-ethyl-4-methanesulfonylphenylacetamide (Example 7, 110 mg, 0.188 mmol) in 1:1 DMF/water (20 mL) was added sodium tungstate (15 mg) followed by 30% aqueous hydrogen peroxide (0.5 mL) dropwise. The resulting mixture was stirred at room temperature for 1 h, diluted with water and extracted with DCM. The organic extracts were washed with water, dried (MgSO₄) and evaporated. The residue was dissolved in ethyl acetate and the solution washed with water, dried (MgSO₄) and evaporated. The residue was purified by eluting through a 20 g BondElut cartridge (gradient elution ethyl acetate to 40% methanol in ethyl acetate) giving the title compound as a solid (80 mg); NMR (d6-DMSO, 120° C.): 1.13 (t, 3H), 1.52 (m, 2H), 1.80 (m, 2H), 1.90 (m, 2H), 1.98 (m, 3H), 2.40 (dd, 2H), 2.89 (m, 5H), 2.99 (m, 4H), 3.14 (s, 3H), 3.31 (q, 2H), 3.80 (s, 2H), 3.80 (m, 1H), 4.80 (dd, 1H), 6.85 (d, 1H), 7.18 (m, 1H), 7.30 (m, 4H), 7.50 (d, 2H), 7.85 (d, 2H); MS: 619 (MH+).

### EXAMPLE 7

This Example illustrates the preparation of (S)-N-[1-(3-phenyl-3-[thiomorpholin-4-ylcarboxyamino]propyl)-4-piperidinyl]-N-ethyl-4-methanesulfonylphenylacetamide (Compound No. 83 of Table I).

To a mixture of (S)-N-[1-(3-phenyl-3-aminopropyl)-4-piperidinyl]-N-ethyl-4-methanesulfonylphenylacetamide dihydrochloride (Method A; 1.3 g, 2.4 mmol) and DIPEA (2.6 mL, 15 mmol) in DCM (50 mL) at 0° C. under argon was added triphosgene (0.3 g, 1.0 mmol) and the resulting mixture stirred for 1 h. 30 mL of this mixture was added to a solution of thiomorpholine (0.15 mL, 1.5 mmol) in DCM (10 mL) and the resulting mixture was stirred at room temperature for 1 h. The mixture was evaporated and the residue partitioned between ethyl acetate and 2M aqueous sodium hydroxide. The organic phase was evaporated and the residue purified by eluting through a 20 g BondElut cartridge (gradient elution ethyl acetate to 30% methanol in ethyl acetate) giving the title compound (105 mg); NMR (d6-DMSO, 120° C.): 1.13 (t, 3H), 1.53 (m, 2H), 1.80 (m, 2H), 1.90 (m, 2H), 1.98 (m, 2H), 2.30 (dd, 2H), 2.50 (m, 4H), 2.89 (m, 2H), 3.14 (s, 3H), 3.31 (q, 2H), 3.66 (m, 4H), 3.80 (s, 2H), 3.80 (m, H), 4.80 (dd, 1H), 6.49 (d, 1H), 7.17 (m, 1H), 7.30 (m, 4H), 7.50 (d, 2H), 7.85 (d, 2H); MS: 587 (ME+).

There now follows NMR data for certain compounds of the invention.

(s)-N-[1-(3-phenyl-3-[2,2-dimethyltetrahydropyran-4-yl-carboxyamino]propyl)-4-piperidinyl]-N-ethyl-4-methanesulfonylphenylacetamide (Compound No. 81 of Table I).

NMR: 1.02 and 1.15 (t, 3H), 1.10 (s, 3H), 1.13 (s, 3H), 1.5 (m, 4H), 1.8 (m, 4H), 2.1 (m, 2H), 2.35 (m; 2H), 2.60 (m, 1H), 2.97 (m, 2H), 3.20 (s, 3H), 3.35 (m, 4H), 3.57 (m, 2H), 3.73 and 4.13 (m, 1H), 3.83 and 3.88 (s, 2H), 4.83 (m, 1H), 7.21 (m, 1H), 7.30 (m, 4H), 7.50 (d, 2H), 7.85 (d, 2H), 8.28 (br s, 1H).

(S)-N-[1-(3-phenyl-3-difluoroacetylaminopropyl)-4-piperidinyl]-N-ethyl-4-methanesulfonylphenylacetamide hydrochloride (Compound No. 84 of Table I).

NMR (d6-DMSO, 120° C.): 1.13 (t, 3H), 1.80 (m, 2H), 2.38 (m, 4H), 2.9-3.1 (m, 4H), 3.14 (s, 3H), 3.35 (q, 2H), 3.47 (m, 2H), 3.89 (s, 2H), 4.21 (m, 1H), 4.98 (dd, 1H), 6.20 (t, 1H), 7.30 (m, 1H), 7.35 (m, 4H), 7.50 (d, 2H), 7.85 (d, 2H), 9.12 (d, 1H), 11.0 (br s, 1H).

(S)-N-[1-(3-phenyl-3-[4-trifluoromethylpyridin-3-ylcarboxyamino]propyl)-4-piperidinyl]-N-ethyl-4-methanesulfonylphenylacetamide hydrochloride (Compound No. 85 of Table I).

NMR (d6-DMSO, 120° C.): 1.15 (t, 3H), 1.80 (m, 2H), 2.38 (m, 4H), 3.0-3.2 (m, 4H), 3.14 (s, 3H), 3.33 (q, 2H), 3.50 (m, 2H), 3.88 (s, 2H), 4.21 (m, 1H), 5.13 (dd, 1H), 7.32 (m, 1H), 7.41 (m, 2H), 7.47 (m, 2H), 7.52 (d, 2H), 7.75 (d, 1H), 7.88 (d, 2H), 8.85 (s, 1H), 8.95 (m, 2H), 10.8 (br s, 1H).

(S)-N-[1-(3-phenyl-3-[3,3,3-trifluoropropionylamino]propyl)-4-piperidinyl]-N-ethyl-4-methanesulfonylphenylacetamide hydrochloride (Compound No. 86 of Table I).

NMR (d6-DMSO, 120° C.): 1.13 (t, 3H), 1.75 (m, 2H), 2.28 (m, 2H), 2.37 (m, 2H), 2.9-3.1 (m, 4H), 3.14 (s, 3H), 3.33 (q, 2H), 3.35 (m, 2H), 3.48 (m, 2H), 3.83 (s, 2H), 4.20 (m, 1H), 4.95 (dd, 1H), 7.25 (m, 1H), 7.35 (m, 4H), 7.50 (d, 2H), 7.85 (d, 2H₁), 8.71 (d, 1H), 11.0 (br s, 1H).

(S)-N-[1-(3-phenyl-3-[3-cyanobenzoylamino]propyl)-4-piperidinyl]-N-ethyl-4-methanesulfonylphenylacetamide (Compound No. 93 of Table I).

NMR: 1.10 and 1.25 (t, 3H), 1.80 (m, 2H), 2.31 (m, 4H), 3.20 (m, 4H), 3.27 (s, 3H), 3.40 (m, 2H), 3.50 (m, 2H), 3.90 and 3.97 (s, 2H), 4.18 and 4.39 (m, 1H), 5.20 (m, 1H), 7.31 (m, 1H), 7.42 (m, 2H), 7.55 (m, 3H), 7.78 (m, 1H), 7.90 (d, 2H), 8.10 (d, 1H), 8.28 (dd, 1H), 8.45 (d, 1H), 8.25 (m, 1H).

(3′S)-N-[1-(1-methyl-3-phenyl-3-[tetrahydropyran-4-yl-carboxyamino]propyl)-4-piperidinyl]-N-ethyl-4-methanesulfonylphenylacetamide (Compound No. 95 of Table I).

NMR: 0.90 (m, 31H), 1.05 and 1.19 (t, 3H), 1.5-2.1 (m, 12H), 2.10 (m, 4H), 2.40 (m, 2H), 2.60 (m, 3H), 2.70 (m, 1H), 3.22 (s, 3H), 3.35 (m, 4H), 3.65 and 3.98 (m, 1H), 3.83 and 3.88 (s, 2H), 4.93 (m, 1H), 7.21 (m, 1H), 7.30 (m, 4H), 7.50 (d, 2H), 7.85 (d, 2H), 8.25 (m, 1H).

(S)-N-[1-(3-phenyl-3-[4,4,4-trifluorobutyrylamino]propyl)-4-piperidinyl]-N-ethyl-4-methanesulfonylphenylacetamide hydrochloride (Compound No. 96 of Table I).

NMR (d6-DMSO, 120° C.): 1.13 (t, 3H), 1.75 (m, 2H), 2.28 (m, 2H), 2.37 (m, 2H), 2.50 (m, 4H), 2.9-3.1 (m, 4H), 3.14 (s, 3H), 3.33 (q, 2H), 3.40 (m, 2H), 3.83 (s, 4H), 4.20 (m, 1H), 4.95 (dd, 1H), 7.25 (m, 1H), 7.35 (m, 4H), 7.50 (d, 2H), 7.85 (d, 2H), 8.40 (d, 1H), 11.0 (br s, 1H).

(S)-N-{1-[3-benzoylamino-3-(3-fluorophenyl)propyl]piperidin-4-yl}-N-ethyl-2-(4-methanesulfonyl-phenyl)acetamide (Compound No. 103 of Table I).

NMR: 1.02 and 1.15 (t, 3H), 1.45 and 1.50 (m, 2H), 1.70 (m, 2H), 1.80 (m, 2H), 1.95 (m, 2H), 2.30 (m, 2H), 2.88 (m, 2H), 3.20 (s, 3H), 3.25 and 3.30 (q, 2H), 3.67 and 4.07 (m, 1H), 3.82 and 3.89 (s, 2H), 5.10 (m, 1H), 7.02 (m, 1H), 7.20 (m, 2H), 7.45 (m, 1H), 7.50 (m, 5H), 7.85 (m, 4H), 8.90 (d, 1H).

(S)-N-{1-[3-(3-fluorobenzoylamino)-3-(3-fluorophenyl)propyl]piperidin-4-yl}-N-ethyl-2-(4-methanesulfonyl-phenyl)acetamide (Compound No. 104 of Table I).

NMR: 1.02 and 1.15 (t, 3H), 1.45 and 1.50 (m, 2H), 1.70 (m, 2H), 1.80 (m, 2H), 1.95 (m, 2H), 2.30 (m, 2H), 2.88 (m, 2H), 3.20 (s, 3H), 3.25 and 3.30 (q, 2H), 3.67 and 4.07 (m, 1H), 3.82 and 3.89 (s, 2H), 5.10 (m, 1H), 7.02 (m, 1H), 7.20 (m, 2H), 7.38 (m, 2H), 7.55 (m, 3H), 7.70 (m, 2H), 7.85 (d, 2H), 8.95 (d, 1H).

(S)-N-{1-[3-(3,3-dimethylbutyrylamino)-3-(3-fluorophenyl)propyl]piperidin-4-yl}-N-ethyl-2-(4-methanesulfonyl-phenyl)acetamide (Compound No. 106 of Table I).

NMR: 0.95 (s, 9H), 1.02 and 1.15 (t, 3H), 1.45 and 1.50 (m, 2H), 1.70 (m, 2H), 1.80 (m, 2H), 1.95 (m, 2H), 2.00 (ABq, 2H), 2.30 (m, 2H), 2.88 (m, 2H), 3.20 (s, 3H), 3.25 and 3.30 (q, 2H), 3.67 and 4.07 (m, 1H), 3.82 and 3.89 (s, 2H), 4.85 (m, 1H), 7.02 (m, 1H), 7.15 (m, 3H), 7.35 (m, 1H), 7.50 (d, 2H), 7.85 (d, 2H), 8.20 (dd, 1H).

(S)-N-[1-(3-phenyl-3-[tetrahydrothiopyran-4-yl-carboxyamino]propyl)-4-piperidinyl]-N-ethyl-4-methanesulfonylphenylacetamide (Compound No. 109 of Table I).

NMR: 1.05 and 1.19 (t, 3H), 1.5-2.1 (m, 12H), 2.30 (m, 2H), 2.65 (m, 4H), 2.90 (m, 1H), 3.22 (s, 3H), 3.35 (m, 4H), 3.73 and 4.13 (m, 1H), 3.83 and 3.88 (s, 2H), 4.83 (m, 1H), 7.21 (m, 1H), 7.30 (m, 4H), 7.50 (d, 2H), 7.85 (d, 2H), 8.25 (m, 1H).

Unless indicated otherwise all the final products were prepared using a method similar to that described for Example 1 using commercially available carboxylic acids with the exception of tetrahydrothiopyrancarboxylic acid (Compound No. 109 of Table 1) which was prepared according to: Helv. Chim. Acta Vol. 80, 1997, 1528-1545.

Starting materials are commercially available, have been described in the literature or can be prepared by adaptation of literature methods. Examples of literature methods include: P. Richter, Ch. Garbe and G. Wagner, E. Ger. Pharmazie, 1974, 29(4), 256-262; C. Oniscu, D. Nicoara and G. Funieru, “4-(Ureidosulfonyl)phenylacetic acid and its ureide”, RO79-966646, (Romanian document); and M. A. Zahran, M. M. Ali, Y. A. Mohammed and A. A. Shehata, Int. J. Chem., 1993, 4(3), 61.

Method A

(S)-N-[1-(3-Phenyl-3-aminopropyl)-4-piperidinyl]-N-ethyl-4-methanesulfonylphenylacetamide dihydrochloride

Step 1: Preparation of 1-phenylmethylethylaminopiperidine Dihydrochloride

To a solution of 1-phenylmethylpiperidone (25.0 g, 132 mmol) in THF (250 mL) was added ethylamine hydrochloride (12.0 g, 147 mmol) and methanol (50 mL) and the resulting mixture stirred at room temperature for 10 min. Sodium triacetoxyborohydride (4 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4ethylaminopiperidine as an oil. This was dissolved in ethanol (500 mL) and concentrated hydrochloric acid (20 mL) was added. The resulting crystals were collected, washed with diethyl ether and dried giving the sub-titled compound as a solid (38 g); NMR (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219 (MH+).

Step 2: Preparation of N-(1-phenylmethyl-4-piperidinyl)-N-ethyl-4-methanesulfonylphenylacetamide

To a solution of 1-phenylmethyl-4-ethylaminopiperidine dihydrochloride (32.0 g, 110 mmol) in DCM (500 mL) was added N,N-diisopropylethylamine (60 mL) with stirring to ensure complete dissolution. 4-Methanesulfonylphenylacetic acid (25.0 g, 117 mmol), 4-dimethylaminopyridine (2.0 g) and dicyclohexylcarbodiimide (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent: 10% MeOH/ethyl acetate) to afford the sub-titled compound (35 g, 76%); NMR: 1.00and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415 (MH+).

Step 3: Preparation of N-(4-piperidinyl)-N-ethyl-4-methanesulfonylphenylacetamide

To a solution of N-(1-phenylmethyl-4-piperidinyl)-N-ethyl-4-methanesulfonylphenyl-acetamide (34 g, 82 mmol) in ethanol (600 mL) was added ammonium formate (40 g). The mixture was purged with argon and 30% Pd on carbon (4.2 g) was added. The resulting. mixture was stirred at reflux for 4 h, then allowed to cool and filtered through diatomaceous earth. The filtrate was evaporated to give a thick oil which solidified on standing to yield the sub-titled compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325 (MH+).

Step 4: Preparation of Title Compound

To a solution of (S)-3-phenyl-3-Boc-aminopropionaldehyde (Method B, 1.4 g, 5.6 mmol) in ethanol (100 mL) and DCM (50 mL) was added N-(4-piperidinyl)-N-ethyl-4-methanesulfonylphenylacetamide (2.0 g, 6.2 mmol), glacial acetic acid (0.6 mL, 10 mmol) and sodium triacetoxyborohydride (2.0 g, 9.4 mmol) and the resulting mixture was stirred at room temperature for 18 h. The mixture was partitioned between DCM and 2M aqueous sodium hydroxide (35 mL), and the organic phase was washed with water, dried and concentrated. the residue was suspended in methanol (10 mL) and concentrated hydrochloric acid (10 mL) was added. The resulting mixture was stirred for 30 min. then evaporated. The residue was azeotroped with ethanol and toluene and triturated with diethyl ether yielding the title compound as a solid (1.3 g); NMR (d6 DMSO at 373K): 1.1 (t, 3H), 1.5 (m, 2H), 1.9 (m, 2H), 2.0 (m, 1H), 2.3 (m, 2H), 3.0 (m, 1H), 3.2 (m, 4H), 3.3 (q, 2H), 3.9 (s, 2H), 4.0 (m, 1H), 4.4 (m, 1H), 7.4 (m, 3H), 7.5 (m, 4H), 7.9 (m, 2H); MS: 458.

Method B

(S)-3-Phenyl-3-Boc-aminopropionaldehyde

To a solution of (S)-N-methyl-N-methoxy-3-phenyl-3-Boc-aminopropionamide (Method C, 5.52 g, 17.9 mmol) in toluene (180 mL) at −20° C. was added sodium bis(2-methoxyethoxy)aluminium hydride (65% solution in toluene, 35.8 mmol) dropwise. The resulting mixture was stirred at −15° C. for 1 h. The mixture was washed with saturated aqueous sodium dihydrogen phosphate solution (250 mL). The organic phase was dried (Na₂SO₄) and concentrated to give the title compound (5 g); NMR: 1.4 (s, 9H), 2.8 (m, 2H), 5.1 (m, 1H), 7.3 (m, 5H), 8.6 (m, 1H), 9.6 (t, 1H).

Method C

(S)-N-Methyl-N-methoxy-3-phenyl-3-Boc-aminopropionamide

To a solution of (S)-3-phenyl-3-Boc-aminopropionic acid (available from PepTech Corp. of Cambridge, Mass., USA; 4.97 g, 18.7 mmol) in DCM (100 mL) was added DIPEA (14.8 mL, 84.8 mmol) and N,O-dimethylhydroxylamine hydrochloride (2.21 g, 22.7 mmol) followed by HATU (8.44 g, 84.8 mmol). The resulting mixture was stirred at room temperature for 18 h, diluted with DCM, washed with 2M aqueous sodium hydroxide and water. The organic phase was dried (Na₂SO₄) and concentrated. The residue was purified by silica column chromatography (eluting with isohexane then 3:1 ethyl acetate to isohexane) giving the title compound as a colourless oil (5.58 g, 97%); NMR (CDCl₃): 1.40 (s, 9H), 2.83 (dd, 1H), 3.01 (m, 1H), 3.08 (s, 3H), 3.52 (s, 3H), 5.10 (m, 1H), 7.28 (m, 5H); MS: 309.

Method D

(4′S)-N-[1-(4-Phenyl-4-aminobut-2-yl)-4-piperidinyl]-N-ethyl-4-methanesulfonylphenylacetamide dihydrochloride

To (4′S)-N-[1-(4-phenyl-4-Bocaminobut-2-yl)-4-piperidinyl]-N-ethyl-4-methanesulfonylphenylacetamide (Method E, 194 mg, 0.339 mmol) was added 5M HCl in methanol (5 mL) and the resulting mixture stirred at room temperature for 3 h. The mixture was evaporated and the residue azeotroped with toluene and triturated with diethyl ether to give the title conpound as a white solid (178 mg, 98%); MS: 472.

Method E

(4′S)-N-[1-(4-Phenyl-4-Bocaminobut-2-yl)-4-piperidinyl]-N-ethyl-4-methanesulfonlIphenylacetamide

To a solution of (S)-4-phenyl-4-Boc-aminobutan-2-one (Method F, 1.25 g, 4.75 mmol) and N-(4-piperidinyl)-N-ethyl-4-methanesulfonylphenylacetamide (1.54 g, 4.75 mmol) in THF/1,2-dichloroethane (1:1, 45 mL) was added titanium tetraisopropoxide (3.1 mL, 10.45 mmol) at room temperature. The resulting mixture was stirred for 15 min. before the addition of sodium triacetoxyborohydride (1.51 g, 7.11 mmol). The resulting mixture was stirred for 18 h before addition of 2M aqueous sodium hydroxide (30 mL). The mixture was diluted with DCM, filtered through Celite®, washed with brine, dried (Na₂SO₄) and concentrated. The residue was purified by BondElut chromatography eluting with a mixture of 1% methanol and 0.05% ammonia in ethyl acetate giving the title compound as a white solid (1.04 g); MS: 572.

Method F

(S)-4-Phenyl-4-Boc-aminobutan-2-one

To a solution of (S)-N-methyl-N-methoxy-3-phenyl-3-Boc-aminopropionamide (Method C, 2.02 g, 6.56 mmol) in THF (70 mL) at −78° C. was added methylmagnesium chloride (3M in THF, 21.1 mmol) dropwise. The resulting mixture was stirred at −78° C. for 30 min. before warning to room temperature over 3 h. The reacton mixture was added to a vigorously stirred mixture of diethyl ether, ice and 1M aqueous potassium dihydrogen phosphate. The aqueous phase was extracted twice with diethyl ether and the combined organic phases washed with sodium hydrogen carbonate solution (sat. aq.) and brine, dried (Na₂SO₄) and concentrated giving the title compound as a white solid (1.27 g, 74%); NMR (CDCl₃): 1.41 (s, 9H), 2.09 (s, 3H), 2.91 (dd, 1H), 3.03 (m, 1H), 5.08 (m, 1H), 5.37 (br s, 1H), 7.28 (m, 5H); MS: 264.

Method G

N-[1-(3-[2-Thienyl]-3-aminopropyl)-4-piperidinyl]-N-ethyl-4-methanesulfonylphenylacetamide

N-[1-(3-[2-Thienyl]-3-Boc-aminopropyl)-4-piperidinyl]-N-ethyl-4-methanesulfonylphenylacetamide (Method H, 0.90 g, 1.6 mmol) was dissolved in trifluoroacetic acid (10 mL) and the resulting mixture stirred at room temperature for 4 h before evaporation. The residue was dissolved in DCM (25 mL) and washed with 2M aqueous sodium hydroxide (2×25 mL), dried and evaporated giving the title compound (470 mg, 63%); NMR: 1.0 (m, 3H), 1.4-2 (m, 7H), 2.3 (m, 2H), 2.9 (m, 2H), 3.2 (s, 3H+H₂O), 3.3 (m, 2H), 3.9 (m, 2H), 4.1 (m, 1H), 6.9 (m, 2H), 7.3 (m, 1H), 7.5 (m, 2H), 7.8 (m, 2H).

Method H

N-[1-(3-[2-Thienyl]-3-Boc-aminopropyl)-4-piperidinyl]-N-ethyl-4-methanesulfonylphenylacetamide

To a mixture of 3-(2-thienyl)-3-Boc-aminopropionaldehyde (Method I, 1.5 g, 5.8 mmol) and N-(4-piperidinyl)-N-ethyl-4-methanesulfonylphenylacetamide (1.9 g, 5.8 mmol) in DCM (20 mL) and ethanol (5 mL) was added one drop of acetic acid. The resulting mixture was stirred at room temperature for 20 min. before the addition of sodium triacetoxyborohydride (1.23 g, 5.83 mmol). The resulting mixture was stirred at room temperature for 18 h. Polymer supported isocyanate resin (1 g) was added and the resulting mixture was stirred at room temperature for 2 h, filtered and eluted through a 10 g SCX cartridge with DCM then methanol then 0.5M ammonia in isopropanol/methanol giving the title compound (0.9 g); NMR: 1.0-1.1 (m, 3H), 1.4 (s, 9H), 1.44 (m, 8H), 2.3 (m, 2H), 2.95 (2m, 2H), 3.3 (s, 3H), 3.9 (d, 2H), 4.8 (m, 1H), 6.9 (m, 2H), 7.3 (d, 1H), 7.5 (m, 3H), 7.8 (m, 2H).

Method I

3-(2-Thienyl)-3-Boc-aminopropionaldehyde

To a solution of 3-(2-thienyl)-3-Boc-aminopropan-1-ol (Method J, 1.5 g, 3.9 mmol) in DCM (50 mL) was added Dess-Martin periodinane (2.5 g, 3.9 mmol) and the resulting mixture was stirred at room temperature for 2 h. The reaction mixture was washed with 2M aqueous sodium hydroxide (2×50 mL), dried and evaporated to give the title compound (1.5 g) which was used in the next reaction without characterisation.

Method J

3-(2-Thienyl)-3-Boc-aminopropan-1-ol

To a solution of 3-(2-thienyl)-3-Boc-aminopropionic acid (2.4 g, 8.85 mmol) in THF (25 mL) was added borane. THF complex (5.9 mL, 1.5M, 8.85 mmol) dropwise. The resulting mixture was stirred at room temperature for 4 h. The mixture was cooled to 0° C. and 2M aqueous sodium hydroxide was added. The mixture was extracted with ethyl acetate (3×50 mL) and the combined extracts dried (MgSO₄) and evaporated giving the title compound (1.5 g) which was used in the next reaction without characterisation.

Method K

N-[1-(3-Cyclohexyl-3-aminopropyl)-4-piperidinyl]-N-ethyl-4-methanesulfonylphenylacetamide

N-[1-(3-Cyclohexyl-3-Boc-aminopropyl)-4-piperidinyl]-N-ethyl-4-methanesulfonylphenylacetamide (Method L, 9.4 g, 20 mmol) was dissolved in trifluoroacetic acid (30 mL) and the resulting mixture was stirred at room temperature for 2 h. Evaporation gave the title compound (3.6 g); NMR: 0.8-1.85 (m, 25H), 2.3 (m, 3H), 2.8 (m, 2H), 3.1 (s, 3H+H₂O), 3.8 (d, 2H), 7.4 (d, 2H), 7.75 (m, 2H).

Method L

N-[1-(3-Cyclohexyl-3-Boc-aminopropyl)-4-piperidinyl]-N-ethyl-4-methanesulfonylphenylacetamide

To a mixture of 3-cyclohexyl-3-Boc-aminopropionaldehyde (Method M, 7 g, 27 mmol) and N-(4-piperidinyl)-N-ethyl-4-methanesulfonylphenylacetamide (9.6 g, 27 mmol) in DCM (200 mL) and ethanol (20 mL) was added acetic acid (0.5 mL). The resulting mixture was stirred at room temperature for 30 min. before the addition of sodium triacetoxyborohydride (5.8 g, 27 mmol). The resulting mixture was stirred at room temperature for 18 h. The reaction mixture was washed with 2M aqueous sodium hydroxide (3×50 mL), dried and evaporated. The residue was purified by silica gel chromatography (eluent: DCM then ethyl acetate then 10% methanol in ethyl acetate) giving the title compound (9.4 g); NMR: 0.8-1.1 (m, 5H), 1.18 (s, 9H), 1.2-2 (m, 11H), 2.2 (m, 2H), 2.8 (m, 2H), 3.3 (s, 3H), 3.8 (d, 2H), 6.5 (d, 1H), 7.5 (m, 2H), 7.8 (m, 2H).

Method M

3-Cyclohexyl-3-Boc-aminopropionaldehyde

To a solution of N-methyl-N-methoxy-3-cyclohexyl-3-Boc-aminopropionamide (Method N, 9.9 g, 31 mmol) in toluene (100 mL) at 0° C. was added sodium bis(2-methoxyethoxy)aluminium hydride (65% solution in toluene, 31 mmol) dropwise. The resulting mixture was stirred at 0° C. for 2 h. 2M aqueous sodium hydroxide was added and the mixture warmed to room temperature and filtered. The filtrate was washed with 2M aqueous sodium hydroxide (2×20 mL), dried and evaporated giving the title compound (7 g) which was used in the next reaction without characterisation.

Method N

N-Methyl-N-methoxy-3-cyclohexyl-3-Boc-aminopropionamide

To a solution of 3-cyclohexyl-3-Boc-aminopropionic acid (Method O, 8.6 g, 32 mmol) and HBTU (12.3 g, 32 mmol) in DMF was added triethylamine (32 mmol) and the resulting mixture was stirred at room temperature for 10 min. N,O-Dimethylhydroxylamine hydrochloride (3.3 g, 32 mmol) was added and the resulting mixture was stirred at room temperature for 18 h before being evaporated. The residue was dissolved in ethyl acetate and the solution washed with water (3×75 mL), dried and evaporated giving the title compound (9.9 g); NMR: 0.8-1.2 (m, 6H), 1.6 (m, 5H), 2.4 (m, 1H), 3 (s, 3H), 3.05 (m, 1H), 3.6 (s, 3H), 3.7 (m, 1H), 6.5 (d, 1H).

Method O

3-Cyclohexyl-3-Boc-aminopropionic Acid

To a mixture of 3-cyclohexyl-3-aminopropionic acid (5 g, 30 mmol), THF (20 mL) and 2M aqueous sodium hydroxide (30 mL, 58 mmol) was added di-tert-butyldicarbonate (9.3 g, 43 mmol) and the resulting mixture was stirred at room temperaure for 8 h. Water (50 mL) was added and the mixture extracted with DCM (2×50 mL). The aqueous phase was acidified to pH 2 and extracted with DCM (5×25 mL). The combined organic extracts were dried and evaporated giving the title compound (8.6 g); NMR: 0.8-1.8 (m, 11H), 2.1-2.4 (m, 2H), 3.6 (m, 1H), 6.6 (d, 1H), 11.95 (s, 1H).

Method P

N-[1-(3-Phenyl-3-methylaminopropyl)-4-piperidinyl]-N-ethyl-4-methanesulfonmlphenylacetamide

This was prepared from 3-phenyl-3-methylaminopropionic acid (Method Q) using a similar sequence of reactions to that used to prepare N-[1-(3-cyclohexyl-3-aminopropyl)-4-piperidinyl]-N-ethyl-4-methanesulfonylphenylacetamide from 3-cyclohexyl-3-aminopropionic acid (Methods O-K).

Method O

3-Phenyl-3-methylaminopropionic Acid

Benzaldehyde (10.6 g, 100 mmol) was added to methylamine (50 mL, 30% in ethanol) and the resulting mixture was stirred at room temperature for 2 h then evaporated. The imine thus formed was dissolved in toluene (100 mL) and malonic acid (10.4 g, 100 mmol) was added. The resulting mixture was heated to 90° C. for 4 h then allowed to cool to room temperature. The solid was collected by filtration to give the title compound (11 g) which was used in the next reaction without characterisation.

Method R

(S)-N-{1-[3-Amino-3-(3-fluorophenyl)propyl]piperidin-4-yl}-N-ethyl-2-(4-methanesulfonyl-phenyl)acetamide

Step 1: Preparation of trans-3-fluorocinnamic Acid tert-butyl Ester

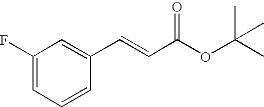

To a stirred solution of trans-3-fluorocinnamic acid (4.34 g, 26. 1 mmol) in toluene (40 mL) at 110° C. was added N,N-dimethylformamide di-tert-butyl acetal (25 mL, 104 mmol) dropwise over 30 min. The resulting mixture was stirred at reflux for a further 4 h. The mixture was then cooled to room temperature and washed with water (50 mL), saturated aqueous sodium hydrogen carbonate solution (2×100 mL) and brine (100 mL), dried (MgSO₄) and evaporated. The crude product was purified by Bond Elut (isohexane then 2% ethyl acetate in isohexane) to give the title compound as a liquid (3.7 g, 64%).

Step 2: Preparation of (s)-3-[(R)-benzyl-(1-phenyl-ethyl)-amino]-3-(3-fluoro-phenyl)-propionic Acid tert-butyl Ester

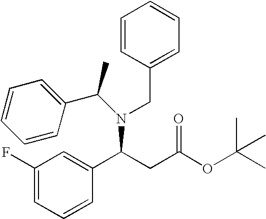

To a stirred solution of (R)-(+)-N-benzyl-α-methylbenzylamine (4.0 mL, 19 mmol) in THP (20 mL) at −78° C. was added n-butyl lithium (1.6M in hexanes, 12.5 mL, 20 mmol) and the resulting mixture was allowed to warm to room temperature over 10 min. before re-cooling to −78° C. A solution of trans-3-fluorocinnamic acid tert-butyl ester (3.74 g, 16.8 mmol) in THF (20 mL) was added and the resulting mixture was stirred at −78° C. for 2 h then quenched by the addition of saturated aqueous ammonium chloride solution (25 mL). After warming to room temperature the organic phase was washed with water (2×50 mL) and brine, dried (MgSO₄) and evaporated. The crude product was purified by Bond Elut (isohexane then 2% ethyl acetate in isohexane) to give the title compound as a gum (5.85 g, 80%); NMR (400 MHz, CDCl₃): 1.23 (s, 9H), 1.27 (d, 3H), 2.48 (m, 2H), 3.67 (s, 2H), 3.97 (q, 1H), 4.40 (dd, 1H), 6.93 (ddd, 1H), 7.1-7.4 (m, 13H).

Step 3: Preparation of 3-tert-butoxycarbonylamino-3-(3-fluoro-phenyl)-propionic Acid tert-butyl Ester

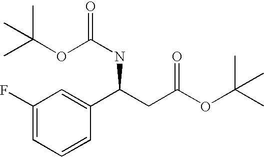

A stirred mixture of (S)-3-[(R)-benzyl-(1-phenyl-ethyl)-amino]-3-(3-fluoro-phenyl)-propionic acid tert-butyl ester (5.39 g, 12.4 mmol), di-tert-butyl dicarbonate (2.98 g, 13.7 mmol) and 20% palladium hydroxide on carbon (0.59 g) in ethanol (100 mL) was hydrogenated at 5 Bar at room temperature for 24 h. The catalyst was removed by filtration through a pad of Celite® washing through with ethanol. The filtrate was evaporated to give an oil which was partitioned between ethyl acetate and saturated aqueous sodium hydrogen carbonate solution. The organic phase was dried (MgSO₄) and evaporated. The crude product was purified by Bond Elut (eluting with isohexane then 5% ethyl acetate in isohexane) to give the title compound as an oil (3.63 g, 86%); NMR: 1.33 (s, 18H), 2.63 (m, 2H), 4.90 (m, 1H), 7.06 (ddd, 1H), 7.24 (m, 2H), 7.37 (dd, 1H), 7.50 (br d, 1H).

Step 4: Preparation of (S)-[1-(3-fluoro-phenyl)-3-hydroxy-propyl]-carbamic Acid tert-butyl Ester

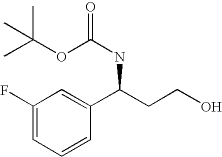

To a stirred, ice-cooled solution of 3-tert-butoxycarbonylamino-3-(3-fluoro-phenyl)-propionic acid tert-butyl ester (2.46 g, 7.25 mmol) in THF (35 mL) was added lithium aluminium hydride (1M in THF, 7.50 mL, 7.50 mmol) dropwise over 20 min. The resulting mixture was stirred with warming to room temperature for 2 h. The reaction was quenched with water (0.275 mL) then 15% aqueous sodium hydroxide (0.275 mL) and more water (0.825 mL) were added with stirring. The resultant precipitate was removed by filtration washing with THF, and the filtrate was dried (MgSO₄) and evaporated. The crude product was purified by Bond Elut (gradient elution, isohexane to 30% ethyl acetate in isohexane) to give the title compound as an oil (1.26 g, 65%); NMR: 1.4 (s, 9H), 1.75 (m, 1H), 1.85 (m, 1H), 3.3 (m, 1H), 3.4 (m, 1H), 4.5 (dd, 1H), 4.65 (br m, 1H), 7.1 (m+br s, 3H), 7.35 (m, 2H).

Step 5: Preparation of (S)-[1-(3-fluoro-phenyl)-3-oxo-propyl]-carbamic Acid tert-butyl Ester

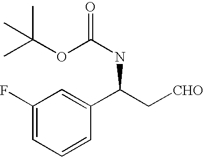

To a solution of (S)-[1-(3-fluoro-phenyl)-3-hydroxy-propyl]-carbamic acid tert-butyl ester (0.85 g, 3.2 mmol) in DCM (70 mL) under argon was added Dess-Martin periodinane (1.48 g, 3.5 mmol) and the resulting mixture was stirred at room temperature for 2 h before the addition of 2M aqueous sodium hydroxide (50 mL). The organic layer was dried (MgSO₄) and evaporated to give the title compound (quantitative); NMR: 1.4 (s, 9H), 2.8 (m, 2H), 5.1 (m, 1H), 7.05 (ddd, 1H), 7.15 (m, 2H), 7.35 (m, 1H), 7.5 (br d, 1H), 9.6 (s, 1H).

Step 6: Preparation of (S)-[3-(4-{ethyl-[2-(4-methanesulfonyl-phenyl)-acetyl]-amino}-piperidin-1-yl)-1-(3-fluoro-phenyl)-propyl]-carbamic Acid tert-butyl Ester

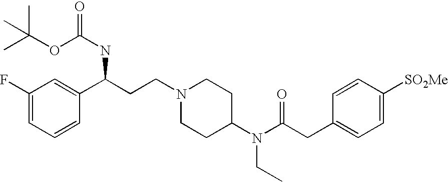

To a solution of (S)-[1-(3-fluoro-phenyl)-3-oxo-propyl]-carbamic acid tert-butyl ester (0.85 g, 3.12 mmol) in DCM (70 mL) and N-ethyl-2-(4-methanesulfonyl-phenyl)-N-piperidin-4-yl-acetamide (Method A, 1.19 g, 3.67 mmol) was added glacial acetic acid (one drop) and the resulting mixture was stirred at room temperature for 1 h. Sodium triacetoxyborohydride (1.4 g, 6.4 mmol) was added and the resulting mixture was stirred at room temperature for 18 h. The reaction mixture was quenched with water and the organic phase was washed with sodium hydrogen carbonated solution (saturated aqueous) and water, dried (MgSO₄) and concentrated. The crude product was purified by Bond Elut (ethyl acetate then 8% methanol in ethyl acetate) to give the title compound as a solid (1.00 g, 55%); NMR: 1.0 and 1.1 (t, 3H), 1.35 (s, 9H), 1.5 (m, 2H), 1.7 (m, 4H), 1.9 (m, 2H), 2.2 (t, 2H), 2.8 (m, 2H), 3.2 (s, 3H), 3.2 and 3.3 (q, 2H), 3.6 and 4.1 (m, 1H), 3.8 and 3.85 (s, 2H), 4.5 (m, 1H), 7.05 (m, 1H), 7.1 (m, 2H), 7.35 (dd, 1H), 7.5 (br d, 1H), 7.5 (d, 2H), 7.85 (d, 2H); LCMS: 576 (MH+).

Step 7: Preparation of Title Compound

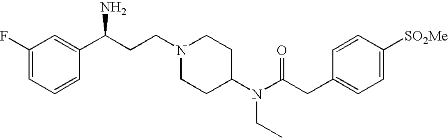

To a solution of (S)-[3-(4-{ethyl-[2-(4-methanesulfonyl-phenyl)-acetyl]-amino}-piperidin-1-yl)-1-(3-fluoro-phenyl)-propyl]-carbamic acid tert-butyl ester (1.00 g, 1.74 mmol) in THF (30 mL) and water (0.1 mL) was added trifluoroacetic acid (5.0 mL) and the resulting mixture was stirred at room temperature for 18 h. The mixture was evaporated and the residue dissolved in DCM. This solution was washed with 2M aqueous sodium hydroxide, dried (MgSO₄) and evaporated to give the title compound (0.84 g, quantitative); NMR: 1.05 and 1.09 (t, 3H), 1.45 and 1.50 (m, 2H), 1.75 (m, 4H), 1.95 (m, 2H), 2.25 (m, 2H), 2.88 (m, 2H), 3.20 (s, 3H), 3.25 and 3.3.0 (q, 2H), 3.67 and 4.08 (m, 1H), 3.82 and 3.89 (s, 2H), 7.00 (m, 1H), 7.15-7.40 (m, 3H), 7.50 (d, 2H), 7.85 (d, 2H), 8.70 (dd, 1H); MS: 476 (MH+).

### EXAMPLE 8

The ability of compounds to inhibit the binding of RANTES was assessed by an in vitro radioligand binding assay. Membranes were prepared from Chinese hamster ovary cells which expressed the recombinant human CCR5 receptor. These membranes were incubated with 0.1 nM iodinated RANTS, scintillation proximity beads and various concentrations of the compounds of the invention in 96-well plates. The amount of iodinated RANTES bound to the receptor was determined by scintillation counting. Competition curves were obtained for compounds and the concentration of compound which displaced 50% of bound iodinated RANTES was calculated (IC₅₀). Preferred compounds of formula (I) have an IC₅₀ of less than 50 μM.

### EXAMPLE 9

The ability of compounds to inhibit the binding of MIP-1α was assessed by an in vitro radioligand binding assay. Membranes were prepared from Chinese hamster ovary cells which expressed the recombinant human CCR5 receptor. These membranes were incubated with 0.1 nM iodinated MIP-1α, scintillation proximity beads and various concentrations of the compounds of the invention in 96 well plates. The amount of iodinated MIP-1α bound to the receptor was determined by scintillation counting. Competition curves were obtained for compounds and the concentration of compound which displaced 50% of bound iodinated MIP-1α was calculated (IC₅₀). Preferred compounds of formula (I) have an IC₅₀ of less than 50 μM.

Results from this test for certain compounds of the invention are presented in Table II. In Table II the results are presented as Pic50 values. A Pic50 value is the negative log (to base 10) of the IC₅₀ result, so an IC50 of 1 μM (that is 1×10⁻⁶M) gives a Pic50 of 6. If a compounds was tested more than once then the data below is an average of the probative tests results.

TABLE II

| Compound No. | Pic50 |
|---|---|
| 61 | 8.2 |
| 62 | 7.01 |
| 53 | 7.81 |
| 63 | 7.04 |
| 64 | 6.48 |
| 52 | 7.85 |
| 68 | 7.18 |
| 79 | 7.55 |
| 82 | 7.7 |
| 83 | 7.97 |
| 85 | 7.32 |
| 88 | 7.48 |
| 89 | 7.24 |
| 90 | 6.61 |
| 91 | 7.52 |
| 92 | 6.2 |
| 93 | 7.79 |
| 94 | 7.56 |
| 95 | 9.72 |
| 97 | 7.57 |
| 98 | 7.84 |
| 99 | 7.39 |
| 100 | 7.63 |
| 101 | 8.33 |
| 102 | 8.02 |
| 103 | 9.36 |
| 104 | 9.35 |
| 105 | 8.54 |
| 106 | 9.25 |
| 107 | 9.03 |
| 109 | 9.27 |
| 81 | 7.7 |
| 84 | 7.45 |
| 86 | 8.7 |
| 87 | 7.79 |
| 96 | 8.63 |
| 108 | 8.02 |
| 110 | 7.34 |

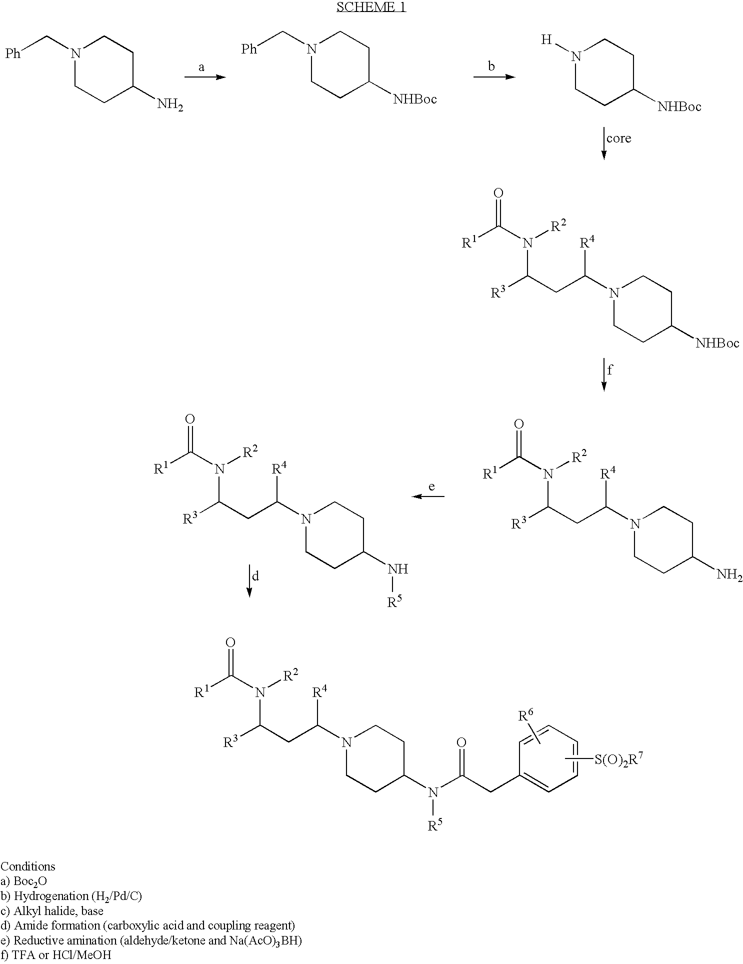

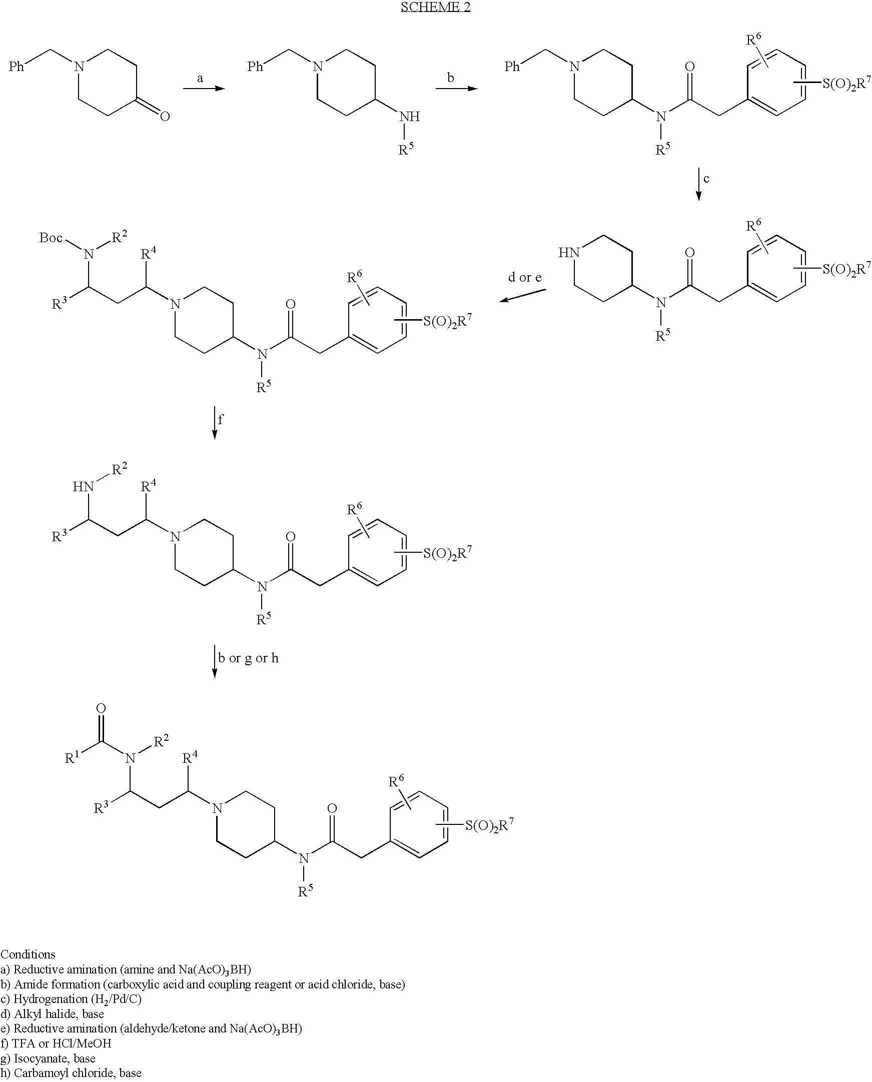

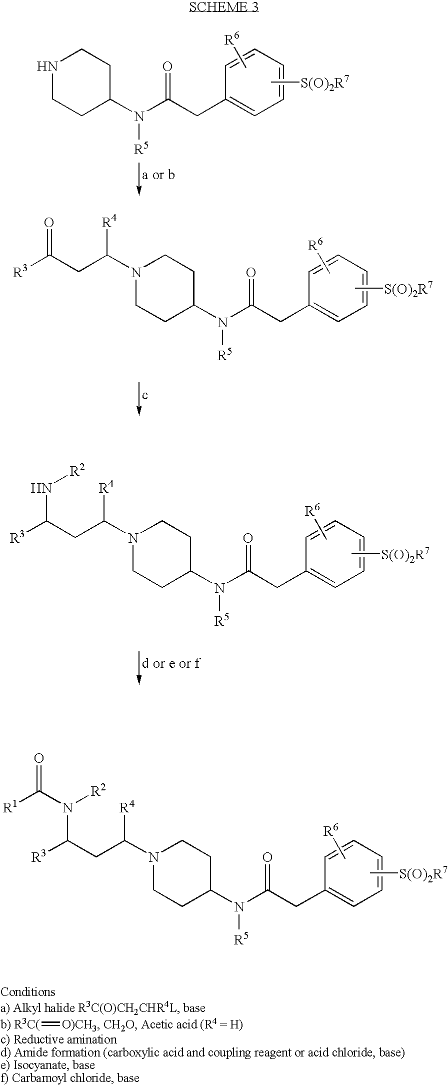

1. A compound of formula (I): 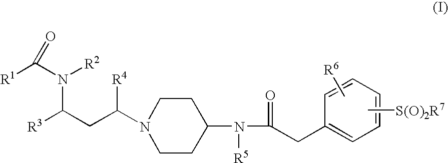
wherein:
R¹ is NHR⁸, C₁₋₆ alkyl {optionally substituted with hydroxy or halo (for example fluoro) or phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, an N-linked 5- or 6-membered non-aromatic heterocyclic ring, or a non-aromatic, 5- or 6-membered mono-heteroatom heterocyclic ring, the heteroatom being oxygen or sulphur {optionally substituted by C₁₋₄ alkyl};
R is hydrogen or C₁₋₆ alkyl;
R³ is phenyl or heteroaryl, either of which is optionally substituted by halo, C₁₋₄ alkyl, C₁₋₄ alkoxy, S(O)ₙ(C₁₋₄ alkyl), nitro, cyano or CF₃; or R³ is C₅₋₇ cycloalkyl;
R⁴ is hydrogen or C₁₋₄ alkyl;
R⁵ is ethyl, allyl or cyclopropyl;
R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
k, m and n are, independently, 0, 1 or 2;
R⁷ is C₁₋₆ alkyl;
R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₃₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
or a pharmaceutically acceptable salt thereof or a solvate thereof; provided that when R¹ is optionally substituted alkyl, optionally substituted phenyl, optionally substituted heteroaryl [wherein heteroaryl is pyrrolyl, imidazolyl, pyrazolyl, 1,2,3-triazolyl, 1,2,4-triazolyl, oxazolyl, isoxazolyl, thiazolyl, isothiazolyl, pyridinyl, pyrimidinyl, pyrazinyl, pyridazinyl, thienyl, furyl, quinolinyl, isoquinolinyl, dihydroisoquinolinyl, indolyl, benzimidazolyl, benzo[b]furyl, benzo[b]thienyl, phthalazinyl, indanyl, oxadiazolyl or benzthiazolyl] or N-linked pyrrolidinyl, and R² and R⁴ are both hydrogen then R³ is not unsubstituted phenyl; and that when R² is hydrogen, R⁴ is methyl and R³ is unsubstituted phenyl then R¹ is not para-chlorophenyl.

2. A compound of formula (I): 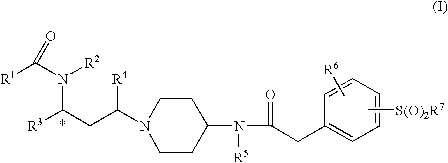
wherein the compounds have the S absolute configuration at chiral center marked with an asterisk ‘*’; and
R¹ is NHR⁸, C₁₋₆ alkyl {optionally substituted with hydroxy or halo (for example fluoro) or phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, heteroaryl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, an N-linked 5- or 6-membered non-aromatic heterocyclic ring, or a non-aromatic, 5- or 6-membered mono-heteroatom heterocyclic ring, the heteroatom being oxygen or sulphur {optionally substituted by C₁₋₄ alkyl};
R² is hydrogen or C₁₋₆ alkyl;
R³ is phenyl or heteroaryl, either of which is optionally substituted by halo, C₁₋₄ alkyl, C₁₋₄ alkoxy, S(O)ₙ(C₁₋₄ alkyl), nitro, cyano or CF₃; or R³ is C₅₋₇ cycloalkyl;
R⁴ is hydrogen or C₁₋₄ alkyl;
R⁵ is ethyl, allyl or cyclopropyl;
R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
k, m and n are, independently, 0, 1 or 2;
R⁷ is C₁₋₆ alkyl;
R⁸ is C₁₋₆ alkyl {optionally substituted with phenyl which is itself optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}, C₃₋₇ cycloalkyl or phenyl {optionally substituted by one or more of: halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
or a pharmaceutically acceptable salt thereof or a solvate thereof.

3. A compound as claimed in claim 1 wherein R¹ is phenyl mono-substituted by fluoro, CF₃, S(O)₂CH₃ or NHS(O)₂CH₃; and R³ is mono-fluoro phenyl.

4. A compound as claimed in claim 1 wherein R¹ is NHR⁸, wherein R⁸ is as claimed in claim 1 or 2, or R¹ is N-linked piperidinyl, N-linked morpholinyl, tetrahydropyran, tetrahydrothiopyran or C₁₋₄ fluoroalkyl having one to six fluorine atoms.

5. A compound as claimed in claim 1 wherein R² is hydrogen.

6. A compound as claimed in claim 1 wherein R³ is phenyl optionally substituted by halo.

7. A compound as claimed in claim 1 wherein R⁴ is hydrogen or methyl.

8. A compound as claimed in claim 1 wherein R⁵ is ethyl.

9. A compound as claimed in claim 1 wherein R⁶ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; and m is 0, 1 or 2.

10. A compound as claimed in claim 1 wherein R⁷ is C₁₋₄ alkyl and wherein the S(O)₂R⁷ group of formula (I) is para disposed to the remainder of the structure of formula (I).

11. Compound No. 1, 2, 3, 4, 5, 6, 7, 8, 9, 10, 11, 12, 13, 14, 15, 16, 17, 33, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 123, 124, 125, 126, 127, 128, 129, 130, 131, 132 or 133 of Table I, or a pharmaceutically acceptable salt thereof or a solvate thereof.

12. A process for the preparation of a compound as claimed in claim 1 the process comprising:
a) coupling a compound of formula (II): 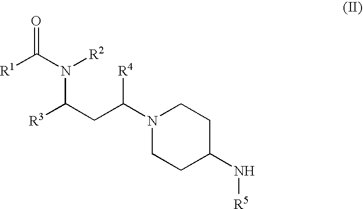
with a compound of formula (III): 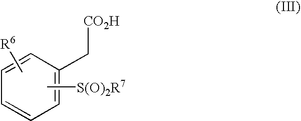
in the presence of a suitable coupling agent, in the presence of a suitable base, in a suitable solvent; or,
b) reacting a compound of formula (IV): 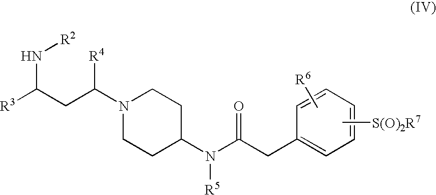
with:
i. an acid of formula R¹CO₂H in the presence of a suitable coupling agent in the presence of a suitable base, in a suitable solvent;
ii. an acid chloride of formula R¹C(O)Cl in the presence of a suitable base, in a suitable solvent;
iii. an isocyanate of formula R¹NCO in the presence of a suitable base in a suitable solvent; or,
iv. a carbamoyl chloride in the presence of a suitable base.

13. A pharmaceutical composition which comprises a compound as claimed in claim 1, or a pharmaceutically acceptable salt thereof or solvate thereof, and a pharmaceutically acceptable adjuvant, diluent or carrier.

14-15. (Cancelled)

16. A method of treating a CCR5 mediated disease state comprising administering to a patient in need of such treatment an effective amount of a compound as claimed in claim 1, or a pharmaceutically acceptable salt thereof or solvate thereof.

17. A pharmaceutical composition which comprises a compound as claimed in claim 2, or a pharmaceutically acceptable salt thereof or solvate thereof, and a pharmaceutically acceptable adjuvant, diluent or carrier.

18. A method of treating a CCR5 mediated disease state comprising administering to a patient in need of such treatment an effective amount of a compound as claimed in claim 2, or a pharmaceutically acceptable salt thereof or solvate thereof.
